(19) United States
(12) Patent Application Publication
(10) Pub. No.: US 2011/0312015 A1
(43) Pub. Date: Dec. 22, 2011

(54) RAPID THROMBOCHEK TEST KIT BASED ON WHOLE BLOOD SCREENING TEST TO DETECT PLATELET HYPERAGGREGATION AT A TEMPERATURE OF 37ºC IN THE CLINICAL LABORATORY

(76) Inventor: DILIP SHRINIVAS VELASKAR, Mumbai (IN)

(21) Appl. No.: 13/111,938

(22) Filed: May 19, 2011

(30) Foreign Application Priority Data
Nov. 19, 2008 (IN) 2440/MUM/2008
Nov. 25, 2008 (IN) PCT/IN2008/000785

(51) Int. Cl. C12Q 1/02

(52) U.S. Cl. 435/29

(57) ABSTRACT

The new RAPID THROMBOCHEK TEST KIT is based on thrombochek screening test for measurement of hyperaggregable platelets in whole blood, which may be present either in form of circulating platelet aggregates, spontaneously aggregating platelets or reacting to weak agonist of platelet aggregation in low concentration—all together, in order to investigate thrombotic tendencies and to assess the efficacy of antithrombotic therapy all measured with help of hematology cell counter and magnetic stirrer at 37° C. of temperature. This is compared to the other existing microscopic method and shows 0.97% correlation. RAPID THROMBOCHEK TEST KIT based on the above methodology is to be used with the aid of an automated hematology analyser and is made available for routine clinical and office laboratory use.

### FIELD OF INVESTIGATION

The present invention relates generally to the methods and measurements for determination of platelet hyperaggregation in a whole blood sample and for detecting and measuring the efficacy of treatment with antiplatelet drugs or the resistance thereto.

### BACKGROUND OF THE INVENTION

Many if not most attacks due to thrombosis can be prevented by timely and appropriate antithrombotic therapy and then also the recurrent episodes can be prevented by appropriate secondary therapy (BARBARA MA).

Most venous and 65% arterial thrombosis are associated with either a blood coagulation protein defect or a platelet function defect, which is either congenital or acquired.

With the current boom in medical technology expansion almost all aeitiologies of hypercoagulation and overt thrombosis are clearly emerging The newer developments in antithrombotic therapy for the treatment of active disorders and for the prophylactic prevention of a thrombotic episode are most promising now than ever before. The use of this fresh information, added on with regular clinical assessment, complimented with wider use of various testing modalities for patient evaluation and the ever increasing influx of newer antithrombotic therapy has lead to conflicting and confusing views amongst the practising physicians and research scientists.

There is scanty knowledge regarding “hyperactive” platelets, more so in that the present literature is one big subject of debate, apart from the single confirmed fact that such a condition definitely exists.

The reason for this lacuna lies mainly in the difficulties involved in setting up, implementing and standardising a proper test method. Most results, in literature, on the hyperactive platelet's are derived by platelet aggregometry, which, in itself, is faulty because it depends on preparation of platelet rich plasma (PRP)′ involves centrifugation of whole blood which leads to loss of sizable population of hyperactive platelets which are in the form of macroplatelets, circulating platelet aggregates or some platelets which may be activated due to centrifugal force forming aggregates. Moreover it depends on measurement of change in turbidity of PRP which may compromise the accuracy of the method. It is time consuming besides being cumbersome and difficult to standardise. The whole blood platelet aggregometry using electrical impedance is more of measurement of platelet adhesion in addition to aggregation. Platelet aggregometry was basically devised to assess platelet function, by hematologists, for their bleeding patients with functional platelet defects. These tests were never invented for the thrombotic patient, although a few technical manipulations exist Yet, hyperactive platelets have been widely described by various techniques in a number of disorders like diabetes mellitus leading to its vascular complication (COLWELL JH ET AL), in unstable angina and atrial fibrillation, thrombotic strokes, migraine headaches, anorexia nervosa, mitral valve prolapse, retinal artery occlusion, pre-eclampsia, arterial thromboembolism and nephrotic syndrome.

Patients in intensive care units have been described as having hyperaggregable platelets. There are some reports of elevated levels of platelet release proteins such as B-thromboblobulin, platelet factor 4 and thromboxane A2 suggesting that the platelets are activated in vivo. It has been so far difficult to establish whether the platelet hyperactivity has been the cause or the effect. Platelet aggregometry has technical difficulties and produces artifacts depending on the aggregometer, technologist and the varied normal ranges for the different agonists (KIRCHOF B ET AL). Probably hyperaggregability is facilitated by the involvement of receptors on the surface of platelet membranes. Association of clinical events and emotional stress is highlighted in many studies and the hyperaggregation response of platelets to adrenaline release or stress is a main point of study especially since it is known that there are platelets with hyperaggregable platelets whose platelet release proteins are not elevated (ROSWELL HC ET AL).

### SUMMARY OF THE INVENTION

Hyperaggregable platelets and their role in thrombogenesis were not well known and little is published on hyperactive platelets, the controversy and lack of information is mainly due to lack of methodology as there is as yet no clinical laboratory screening test for hyperaggregable platelets, while aggregometry, so far used, was basically designed for studying bleeding disorders rather than thrombotic disease (FRENKEL EP ET AL).

The recent flow cytometric studies, though adding valuable information, can hardly be described as an affordable screening test for platelets.

The present invention is a first of its kind, an affordable screening test, for detection of all hyperactive platelets, in the office laboratory or point of care units and is devised primarily from our own observations and studies:

a), that EDTA Whole blood gives single platelet count since EDTA brakes all the existing platelet aggregates.
b), that agonist treated stirred citrate whole blood may give low platelet count which may be due to presence of Circulating platelet aggregates or hyperactive platelets forming spontaneous platelet aggregates due to stirring or platelet reacting with weak agonist of platelet aggregation in low concentration.
c), that calculation of percentage difference of EDTA platelet count and weak agonist in low concentration treated stirred citrated platelet count gives absolute measure of hyperactive platelet aggregation.

A first aspect of this invention relates to the methods of demonstrating hyperactive platelets by:
1. Collecting a blood sample from an individual in EDTA anticoagulant vacutainer first and then taking blood in trisodium citrate-anticoagulant vacutainer with the same venipuncture and mixing both the samples with the respective anticoagulants at least 15 to 20 times by inversion and allowing EDTA sample to stand at room temperature for a minimum of 20 minutes and then within two hours of collection, the EDTA sample is analysed on an automated hematology analyser to determine the platelet count and appropriate aliquote of citrate sample is taken in a plastic cuvette with a magnetic bar and stirred for 3 minutes at 37° C. and a weak agonist of platelet aggregation in low concentration is added to stirred citrate sample and further stirred for another three minutes at 37° C. At the end of these last three minutes the stirred citrate agonist treated sample is analysed on an hematology analyser for platelet count. Difference between EDTA platelet count and agonist treated stirred citrate platelet count, percentage of platelet aggregation is calculated by dividing the difference by EDTA platelet count and multiplying it by 100.

This platelet aggregation may be due to presence of either circulating platelet aggregates, spontaneously aggregating platelets due to stirring or platelets reacting to weak agonist in low concentration or all of these together.

### DETAILED DESCRIPTION OF THE INVENTION

The new test relates to measuring platelet hyper-aggregation in the view of investigating thrombotic tendencies and to form its treatment modalities and subsequent followup:—

It can be studied in mammals—primates such as humans, chimpanzees, dogs, pigs etc. Blood is obtained by venipuncture using preferably precisionglyde (Becton Dikinson) it can be collected during a medically invasive procedure, such as the open heart procedure Blood sample is collected by vaccutainer system using the following collection procedure: the vaccutainers connected to veinipuncture needle in the following order, first the plain vaccutainer, which, if not required for other tests, is to be discarded then the EDTA vaccutainer and thirdly the Na citrate 3.8% is to be connected to the vaccutainer needle. The samples collected in EDTA and 3.8% Na citrate (1 in 10 dil) anticoagulant vacutainers are immediately and thoroughly mixed by inversion (15 to 20 times) and then allowed to stand at room temperature for a minimum period of 20 minutes before starting the test. Twenty minutes is the time given for the EDTA to act on existing platelet aggregates to break down and hence give the single platelet count.

Heparin, Na Citrate 3.2% can also be used instead of citrate 3.8% as anticoagulant to study platelet hyperaggregation. D-Phe-Pro-Arg chloromethyl ketone dihydrochloride used as an anticoagulant in some studies is not considered here as an anticoagulant for this study because there is substantial disaggregation of platelet aggregation caused by addition of agonist creating the doubt whether D-Phe-Pro-Arg chloromethyl ketone dihydrochloride has antiplatelet action.

Analysis of platelet counts of the blood samples can be carried out on any hematology analyser preferably automated impedance type of hematology analyser, eg: the Coulter counter. This hematology analyser is to be used for identifying single platelet count on EDTA samples platelet aggregation as a result of changes in the platelet counts caused by stirring of the blood sample on a magnetic stirrer followed by addition of agonist causing platelet aggregation. The magnetic stirrer with inbuilt 37° C. incubator tailor-made to hold flat bottom plastic tubes with magnetisable metal bar with non-water-wettable surface use here for stirring is from Velaskar Laboratories, exclusively devised for this test as an affordable accessory.

Agonist available for inducing platelet aggregation are Adenosine diphosphate (ADP), epinephrine bitartrate, collagen, thrombin, etc. Epinephrine is the preferred agonist as it is released in stress.

In carrying out the method of current invention, the hyperactive platelet aggregation is measured by analysing the EDTA sample, after thoroughly mixing by inversion after collection and allowing to stand at room temperature for twenty minutes, on hematology analyser. Simultaneously, after proper mixing, an aliquote of citrate sample is taken in a plastic cuvette with metal bar, stirred on the magnetic stirrer at 37° C. for 180 seconds and then again stirred for another 180 seconds after audition of weak agonist at 37° C., and also analysed on the hematology analyser and the difference between EDTA platelet count and agonist treated stirred citrate sample platelet count divided by the EDTA platelet count and multiplied by 100 gives the percentage of hyperactive platelets which may be due to presence of either circulating platelet aggregates, spontaneously aggregating platelets or platelets reacting to weak agonist of platelet aggregation.

Tests were carried out on healthy adults (male and female) and the normals were established. The test was carried out on patients of diabetes mellitus and known cases of myocardial infarction. Simultaneously blood smears were prepared at each step and stained and examined to correlate with the microscopic method for platelet aggregation, a work of study published in 1982, for measurement of hyperactive platelets (5)(VELASKAR DS ET AL). A strong correlation is obtained between the two methods and the present invention is found to be best suited for the demonstration of hyperaggregable platelets in investigating thrombotic tendencies and for its subsequent treatment and followup A group of patients on anti-platelet—Aspirin therapy were tested by new Rapid Thrombochek test for assessment of efficacy.

### Material and Methods:

Four groups of individuals were examined:
Group 1: Twenty healthy adult men and twenty healthy adult women between the ages of 22 to 35 years were tested appropriately as detailed
Group 2: Twenty cases of diabetes mellitus (fasting plasma glucose levels of more than 130 mg/dl were tested appropriately as detailed
Group 3: Twenty cases of myocardial infarction were tested appropriately as detailed
Group 4: Twenty patients on Aspirin for minimum period of 3 weeks.

### Equipment and Reagents:

B D vacutainers for EDTA, 3.8% trisodium citrate and 20 G needles, 8/30 mm flat bottomed plastic tubes, Magnetic stirrer with inbuilt incubator for 37° C. (Velaskar), magnetisable metal bar with non-water-wettable surface, automated hematology analyser (Coulter-Impedance type particle counter), Glass slides, automated Pipettes, stop watch and microscope with oil immersion lens. Reagents used: Epinephrine bitartrate 0.5 uM, 2.5 uM as agonist of platelet aggregation, alcohol and Wright-Giemsa's stain. Blood samples for Analysis.

All samples were collected in the morning when the subjects were fasting. Blood was collected in Plain vaccutainer then EDTA vacutainer by a clean venipuncture using a 20 G needle and then in the 3.8% Na citrate (1:10 dil) vacutainer. Both these anticoagulated blood samples were thoroughly mixed with respective anticoagulants by inversion (15 to 20 times) and the EDTA sample was made to stand at room temperature for 20 minutes after collection and only then analysed for platelet count measurements on the automated hematology analyser. Both samples were analysed within two hours of collection.

### Procedure:

STEP 1: EDTA sample analysed on hematology analyser after repeated mixing by inversion, 20 minutes after collection and platelet count recorded
STEP 2: An aliquot of the citrated sample is transferred to a flat bottomed plastic tube (8/30 mm) with a magnetisable metal bar with non-water-wettable surface and then stirred on a magnetic stirrer with 37° C. incubation, stirring continuously at a speed of 1000 rpm for 3 minutes
STEP 3: To the stirred aliquot of citrated sample in the plastic tube an agonist was added, the final concentration being 0.5 uM and the stop watch was started while the sample was stirred continuously for 180 sec at 37° C. Exactly at the end of these 180 sec the sample was analysed for platelet count on hematology analyser and this was recorded. Simultaneously a blood smear from the same sample was prepared and marked.

### Examination and Recording:

Percentage of hyperaggregating platelets, which may be either circulating platelet aggregates, spontaneously aggregating platelets or platelets reacting to weak agonist of platelet aggregation at 180 seconds; was calculated by finding out the difference between EDTA platelet count and platelet count of agonist treated stirred citrate sample at 180 seconds and dividing it by EDTA sample platelet count and then multiplying it by 100.

All the platelet counts where dilutional effect was observed due to anticoagulant or agonist were corrected.

### Comparative Studies

All the subjects were simultaneously examined by MICROSCOPIC METHOD (5) of studying platelet aggregation as follows:

For measuring hyperactive platelet aggregation having either circulating platelet aggregates, spontaneously aggregating platelets or platelets reacting to weak agonist, a smear was prepared from the agonist treated stirred agonist treated citrated sample of blood at 180 seconds.

All the smears were prepared by taking a drop of blood on slide and making a smear by cover slide method of smear preparation. All the smears were fixed in alcohol after drying and stained with WRIGHT-GEIMSA stain and examined under oil immersion lense. Platelets free and in aggregates were counted and percentage of aggregation was worked out.

### Results

Results obtained in all the three groups of subjects for platelet aggregation either having circulating platelet aggregates, spontaneously aggregating platelets or platelets reacting to weak agonists of platelet aggregation (0.5 uM Epinephrine final concentration) at 180 seconds showing Mean value and Standard deviation as depicted in Table 1.

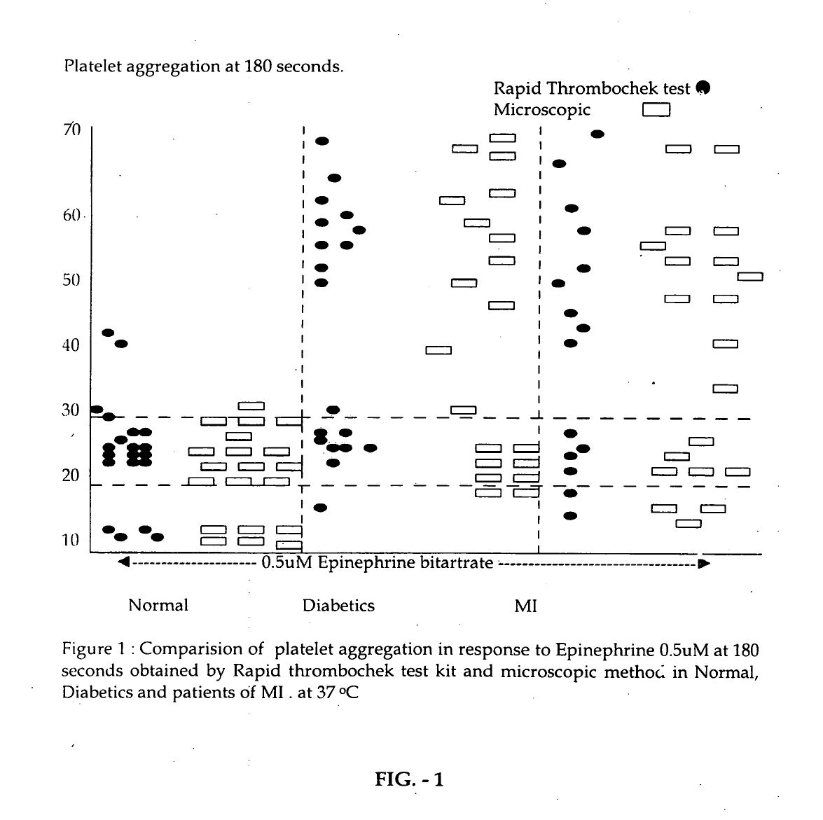

Comparison of platelet aggregation in response to Epinephrine 0.5 uM at 180 seconds obtained by RAPID Thrombochek test and Microscopic method in Normals, Diabetics and Myocardial Infarction in FIG. 1.

TABLE 1

| | Platelet Aggregation % |
|---|---|
| Group | at 180 sec. at 37° C. |
| Normal Range | 7 to 22 |
| Mean | 14.5 (7) |
| MI Range | 18 to 40 |
| Mean | 29 (11) |
| Diabetes Range | 23 to 48 |
| Mean | 35 (12) |

The table shows Mean values and figures in bracket shows Standard Deviation.
Platelet aggregation at 180 seconds after the addition of 0.5 uM Epinephrine Expressed as percentage. (By RAPID THROMBOCHEK TEST).

TWENTY PATIENTS OF MYOCARDIAL INFARCTION ON DAILY ASPIRIN FOR MINIMUM PERIOD OF 3 WEEKS WERE TESTED BY RAPID THROMBOCHEK TEST AS WELL AS BY MICROSCOPIC METHOD FOR ASSESSMENT OF EFFICACY OF ANTIPLATELET TREATMENT. THE RESULTS ARE AS FOLLOWS: FOUR PATIENTS SHOWED PRESENCE OF HYPERACTIVE PLATELETS WAS ABOVE NORMAL RANGE BY BOTH THE METHODS INDICATING THAT ANTI-PLATELET THERAPY WAS LESS THAN EFFECTIVE IN FOUR OF THEM.

### CONCLUSIONS

1. THE RAPID THROMBOCHEK TEST IS BASED IN THE CLINICAL LABORATORY SETTING AND IS AN EFFICIENT AND RAPID SCREENING TEST FOR EARLY DETECTION OF PLATELET HYPER AGGREGATION IN THROMBOTIC DISEASE, THEREFORE CAN BE USED IN PREVENTION, ESPECIALLY IN DIABETIC PATIENTS WITH MYOCARDIAL INFARCTS AND IN SMOKERS, IN HYPERTENSIVES, HYPER LIPIDEMICS; IT CAN BE USED FOR DOSAGE MONITORING FOR PATIENTS ON ANTIPLATELET THERAPY
2. THE RAPID THROMBOCHEK TEST COMPARES WELL WITH THE KNOWN AND ESTABLISHED MICROSCOPIC METHOD OF MEASURING PLATELET HYPERAGGREGATION
3. THE THROMBOCHEK TEST CAN EASILY BE USED AT POINT OF CARE WITH APPROPRIATE ANTICOAGULANT FOR BLOOD SAMPLE COLLECTION FOR PATIENTS ON THERAPY, FOR MEDICAL MANAGEMENT OF NON-ST SEGMENT ELEVATION ACUTE CORONARY SYNDROMES AND ESPECIALLY PATIENTS TREATED WITH PER CUTANEOUS CORONARY INTERVENTION

### REFERENCES

1). BARBARA MA; SEM HEMAT 2002; 39:143-144
2). COLWELL JH ET AL: MED CLIN NORTH AM 1978; 62; 753-756
3). FRENKEL EP ET AL: HEMATOL ONCOL CLIN N AM 17 (2003) 63-83
4). ROSWELL H C ET AL BR J HAEMAT 1966; 12:66-71S
5). VELASKAR DS ET AL: AM J. CLIN. PATH 1982; 77:3:267-274

We claim:

1. A rapid screening method for demonstration of hyperactive platelets to identify population prone to atherosclerosis and its prevention:—
a) screening test based on observation that a agonist treated stirred citrated whole blood sample shows low platelet count due to presence of platelet aggregates as against a EDTA whole blood sample gives a single platelet count as all the existing platelet aggregates are broker down.
b) A percentage difference of in EDTA platelet count and a weak and low concentration agonist treated stirred citrate platelet count gives a measure of presence of hyperactive platelets.
c) Hyperactive platelets can be either in the form of either circulating platelet aggregates or spontaneously aggregating platelets due to stirring or platelets reacting to low concentration of weak agonist of platelet aggregation.
d) The blood sample is collected in EDTA and in 3.8% sodium citrate (1 in 10)
vaccutainer, after proper mixing by inversion with respective anticoagulants and allowing both the samples to stand at room temperature for minimum of 20 minutes allowing EDTA to break existing platelet aggregates and analysing the EDTA sample on hematology cell counter for platelet count-and transferring appropriate aliquote of citrated blood sample to flat bottom plastic cuvette with magnetisable metal bar with non-water-wettable surface and stirring it in magnetic stirrer with 37° C. incubator tailor-maid to hold the cuvettes for 180 seconds on a magnetic stirrer at 1000 rpm and at 37° C. temperature, exposing the same stirred sample to a weak agonist of platelet aggregation in its low concentration and further stirring for suitable time, ideally 180 seconds, at 1000 rpm speed and at 37° C. and immediately analysing the same agonist treated citrate sample on hematology cell counter for its platelet count and then calculating the difference in EDTA platelet count and agonist treated stirred citrate platelet count to give percentage of platelet aggregation at end of this time, ideally 180 seconds, which may be due to presence of either circulating platelet aggregates, spontaneously aggregating platelets or platelets reacting to weak agonist of platelet aggregation by dividing the difference in platelet counts by EDTA platelet count and then multiplying it 100 and to analyse blood samples obtained in the same above manner from statistically significant normal healthy individuals by the same above method, to establish a normal range of platelet aggregation having either circulating platelet aggregates, spontaneously aggregating platelets or platelets reacting to weak agonist of platelet aggregation at 180 seconds by calculating their mean and standard-deviation.

2. The method according to claim 1 of collecting blood samples in serial vacutainers EDTA vaccutainer first and followed by citrate vaccutainer and mixing the same by inversion for at least 15 to 20 times, in their corresponding anticoagulant and tested within 3 hrs after collection.

3. The method according to claim 1 of letting the EDTA sample to stand at room temperature for a minimum of 20 minutes so as to obtain single platelet count after allowing EDTA to break existing platelet aggregates and allowing the citrate sample to stand at room temperature for the same time

4. The method according to claim 1, wherein Epinephrine bitartrate is termed a weak agonist since platelets from about 20% of population do not show aggregation with Epinephrine bitartrate which itself needs further investigation as to whether this population has protection against athersclerosis!

5. The method according to claim 1 of using a weak agonist of platelet aggregation for exposure to stirred citrate sample, this being Epinephrine bi tartrate 0.5 uM final concentration.

6. The method according to claim 1 of calculating percentage of platelet aggregation at suitable time, ideally 180 sec, of stirring and 180 secs with exposure to weak agonist by calculating the difference between EDTA platelet count and agonist treated stirred citrated sample platelet count at the end of this suitable time, ideally 180 seconds, multiplying it by 100, and allowing 0.97% correlation to the microscopic method (5) of platelet aggregation, described in the year 1982 and used in many clinical studies as a basic study.

7. The method of determining or monitoring the efficacy of antiplatelet therapy comprising of obtaining a blood sample, from an individual treated for anti-platelet therapy, in EDTA vaccutainer and in trisodium Citrate 3.8% vaccutainer, allowing both the samples to stand at room temperature for minimum of 20 minutes and analysing EDTA sample on hematology cell counter for platelet count-and transferring appropriate aliquote of citrated blood sample to a flat bottom plastic cuvette with magnetisable metal bar with non-water-wettable surface and stirring it for 180 seconds on a magnetic stirrer at 1000 rpm and at 37° C. temperature, exposing the same stirred sample to an agonist of platelet aggregation in its suitable concentration and further stirring for suitable time, ideally 180 seconds, at 1000 rpm speed and at 37° C. and immediately analysing the same agonist treated citrated sample on hematology cell counter for its platelet count within 2 hours of collection of samples, and then calculating the difference in EDTA platelet count and agonist treated stirred citrated platelet count to give percentage of platelet aggregation at the end of suitable time, ideally 180 seconds, which may be due to presence of either circulating platelet aggregates, spontaneously aggregating platelets or platelets reacting to a weak agonist of platelet aggregation by dividing the difference in platelet counts by EDTA platelet count and then multiplying it by 100 which may give percentage measure of hyperactive platelets in the form of either Circulating platelet aggregates, Spontaneously aggregating platelets or platelets hyperreacting to a weak agonist of platelet aggregation at suitable time, ideally 180 seconds, all together, to determine the platelet aggregation inhibition wherein presence of hyperactive platelets is at or above normal range indicating that antiplatelet therapy is less than effective.

8. The method according to claim 7 where agonist used is Epinephrine bitartarate and 2.5 uM is the final concentration.

9. The RAPID THROMBOCHEK TEST KIT for routine clinical laboratory, office laboratory or point of care units, based on the modified thrombochek test methodology described in the above claims, comprising of a suitable platelet agonist reagent in any appropriate concentration, a diluting fluid for the reagent and a flat bottom plastic tube containing a magnetisable metal bar with non-water-wettable surface along with instructions for application to measure platelet hyperaggregation in whole blood which may be in the form of either 1). circulating platelet aggregates and, or 2). causing spontaneous platelet aggregation and or 3). hyper-reacting to a weak agonist of platelet aggregation, all to be measured with the help of an automated hematology analyser and a magnetic stirrer with 37° C. incubator tailor-made to hold the flat bottom plastic tube with magnetizable metal bar supplied.

10. The Thrombochek test kit according to claim 9, is provided with a tailor-made Magnetic stirrer with 37° C. temperature incubator to hold flat bottomed plastic tubes with magnetisable metal bars which is with nonwaterwettable surface.

11. The Thrombochek test kit according to claim 9, wherein, the magnetic stirrer with incubator maintains temperature of agonist treated stirred citrate sample of blood at 37° C.

12. The Thrombochek test kit according to claim 9, wherein the magnetic stirrer with Incubator has inbuilt controls to monitor the temperature and speed of stirring.

13. The Thrombochek test kit according to claim 9, is provided with disposable Plastic tubes containing, magnetisable metal bar with nonwaterwettable surface, for correct stirring, the plastic flat bottomed tube meant to fit in and work with the tailormade magnetic stirrer provided with the test kit.

14. The Thrombochek test kit according to claim 9, wherein, the magnetisable metal bars are coated with silicone or a silicone like substance to make its surface non-water wettable.

15. The Thrombochek test kit according to claim 9, wherein the test kit is provided with a detailed procedure manual of instructions of methodology.
